(19) United States
(12) Patent Application Publication
(10) Pub. No.: US 2018/0340946 A1
(43) Pub. Date: Nov. 29, 2018

(54) METHOD OF ANALYZING BIOMATERIAL

(71) Applicant: ELECTRONICS AND TELECOMMUNICATIONS RESEARCH INSTITUTE, Daejeon (KR)

(72) Inventors: Young Jun KIM, Daejeon (KR); Wan Joong KIM, Daejeon (KR); Eun Hye JANG, Sejong (KR); HyoYoung CHO, Daejeon (KR)

(21) Appl. No.: 15/871,679

(22) Filed: Jan. 15, 2018

(30) Foreign Application Priority Data
May 23, 2017 (KR) 10-2017-0063737

(51) Int. Cl. G01N 33/74; G01N 33/566; C12Q 1/28; G01N 33/52; G01N 33/543

(52) U.S. Cl. CPC G01N 33/74; G01N 33/566; G01N 33/54353; G01N 33/52; C12Q 1/28

(57) ABSTRACT

According to the inventive concept, a method of analyzing a biomaterial may include preparing an analysis apparatus including a substrate having a first region and a second region, supplying a second antigen onto the substrate to conduct a first reaction of a portion of antibodies and the second antigen, and conducting a second reaction of another portion of the antibodies and a first antigen after conducting the first reaction, to form a binding structure. The antibodies may be disposed in the first region of the substrate, the capturing structure may be provided in the second region of the substrate, and the capturing structure may include a linker which binds to the substrate and the first antigen which binds to the linker. The binding structure may include the linker, the first antigen and the antibody which binds to the first antigen.

### CROSS-REFERENCE TO RELATED APPLICATIONS

This U.S. non-provisional patent application claims priority under 35 U.S.C. § 119 of Korean Patent Application No. 10-2017-0063737, filed on May 23, 2017, the entire contents of which are hereby incorporated by reference.

### BACKGROUND

The present disclosure herein relates to a method of analyzing a biomaterial, and more particularly, to a method of analyzing a biomaterial using antigen-antibody reaction.

Bio-technology (BT) is one kind of next-generation fusion technologies, and its importance is increasing. Recently, research on the analysis of biomaterials is increasing. The biomaterials may be supplied in small quantity. Biomaterials may have a small molecular weight. For example, the biomaterials related to hormones may have a small molecular weight. Accordingly, there is growing need to analyze biomaterials with a small molecular weight. In addition, the requirement on methods of accurately analyzing biomaterials is increasing.

Biomaterials may include antigens and antibodies. The antibodies may specifically bind to the antibodies. Recently, for the analysis of biomaterials, research on methods using antigens and antibodies is being conducted.

### SUMMARY

The technical task for solving in the present disclosure is providing a method of analyzing a biomaterial having a small molecular weight.

Another technical task for solving in the present disclosure is providing a method of analyzing a biomaterial with improved sensitivity and accuracy.

The tasks for solving in the present disclosure are not limited to the above-described tasks, and non-referred other tasks may be clearly understood from the description below by a person skilled in the art.

An embodiment of the inventive concept relates to a method of analyzing a biomaterial. According to the inventive concept, the method of analyzing a biomaterial includes preparing a substrate including a first region and a second region, where antibodies are disposed in the first region of the substrate, a capturing structure is provided in the second region of the substrate, and the capturing structure includes a linker which binds to the substrate and a first antigen which binds to the linker, supplying a second antigen onto the substrate to conduct a first reaction of the second antigen and a portion of the antibodies, and conducting a second reaction of the first antigen and another portion of the antibodies after conducting the first reaction, to form a binding structure, wherein the binding structure includes at least one of the linker, the first antigen or the antibodies.

In an embodiment, the method may further include supplying a colorimetric-material solution onto the substrate to form a colored product, supplying light onto the substrate, and analyzing light absorbed by the colored product.

In an embodiment, the antibodies may have labels, and the labels may include a peroxidase enzyme.

In an embodiment, the colorimetric-material solution may include 3,3′,5,5′-tetramethylbenzidine and hydrogen peroxide (H₂O₂).

In an embodiment, the first antigen may include at least one of cortisol or cortisol derivatives, and the second antigen may include at least one of cortisol or cortisol derivatives.

In an embodiment, the linker may be represented by the following Formula 1:

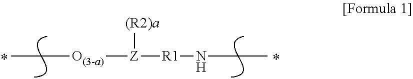

(in Formula 1, Z is one selected among silicon (Si) and carbon (C), R₁ includes at least one selected from —(CH₂)ₙ—, —(CH₂)ₘ—(CH₂CH₂)ₙ—, and —(CH₂)ₘ—(NH—CH₂CH₂)ₙ—, R₂ is a substituted or unsubstituted alkyl group having 1 to 5 carbon atoms, “a” is an integer selected among 0, 1 and 2, “n” is an integer selected from 1 to 10, and “m” is an integer selected from 0 to 10.)

In an embodiment of the inventive concept, a method of analyzing a biomaterial includes preparing an analysis apparatus including a filter and a substrate, where antibodies are provided in the filter, a capturing structure is provided on the substrate, and the capturing structure includes a linker which binds to the substrate and a first antigen which binds to the linker, supplying second antigens into the filter to conduct a first reaction of the second antigen and a portion of the antibodies, moving another portion of the antibodies onto the substrate after conducting the first reaction, and forming a binding structure via a second reaction of the first antigen and another portion of the antibodies, wherein the binding structure includes at least one of the linker, the first antigen, or the antibodies.

In an embodiment, the method may further include supplying a colorimetric-material solution onto the substrate to form a colored product, supplying light onto the substrate, and analyzing light absorbed by the colored product to quantitatively analyze the second antigen.

In an embodiment, the first antigen may include at least one of cortisol or cortisol derivatives, and the second antigen may include at least one of cortisol or cortisol derivatives.

In an embodiment, the linker may be represented by the following Formula 1:

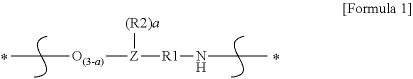

(in Formula 1, Z is one selected among silicon (Si) and carbon (C), R₁ includes at least one selected from —(CH₂)ₙ—, —(CH₂)ₘ—(CH₂CH₂)ₙ—, and —(CH₂)ₘ—(NH—CH₂CH₂)ₙ—, R₂ is a substituted or unsubstituted alkyl group having 1 to 5 carbon atoms, “a” is an integer selected among 0, 1 and 2, “n” is an integer selected from 1 to 10, and “m” is an integer selected from 0 to 10.)

In an embodiment, the substrate may include a well plate or a capillary.

### BRIEF DESCRIPTION OF THE FIGURES

The accompanying drawings are included to provide a further understanding of the inventive concept, and are incorporated in and constitute a part of this specification. The drawings illustrate exemplary embodiments of the inventive concept and, together with the description, serve to explain principles of the inventive concept. In the drawings:

FIG. 1 is a diagram schematically illustrating an analysis apparatus according to exemplary embodiments;

FIG. 2 is a cross-sectional view illustrating a detection part of an analysis apparatus according to exemplary embodiments;

FIGS. 3A and 3B are cross-sectional views illustrating a method of manufacturing an analysis apparatus according to exemplary embodiments;

FIGS. 4A to 4E are cross-sectional views illustrating a method of analyzing a biomaterial according to exemplary embodiments;

FIG. 5 is a cross-sectional view for explaining an analyzing method according to another embodiment;

FIG. 6 is a cross-sectional view illustrating a detection part of an analysis apparatus according to exemplary embodiments;

FIGS. 7A to 7E are cross-sectional views illustrating a method of analyzing a biomaterial according to exemplary embodiments; and

FIGS. 8A to 8C illustrate the measured results of absorbance using linkers represented by Formula 5a to Formula 5c, respectively.

### DETAILED DESCRIPTION

Hereinafter, preferred embodiments of the inventive concept will be explained in detail with reference to the accompanying drawings for the sufficient understanding of the configuration and effects of the inventive concept. The inventive concept may, however, be embodied in different forms and should not be construed as limited to the embodiments set forth herein. Rather, these embodiments are provided so that this disclosure will be thorough and complete, and will fully convey the scope of the inventive concept to those skilled in the art. One of ordinary skill in the art will understand appropriate circumstances in which the concept of the present disclosure may be conducted.

The terminology used herein is for the purpose of describing particular embodiments only and is not intended to be limiting of the invention. As used herein, the singular forms are intended to include the plural forms as well, unless the context clearly indicates otherwise. It will be further understood that the terms “comprises” and/or “comprising”, when used in this specification, specify the presence of stated components, steps, operations and/or elements, but do not preclude the presence or addition of one or more other components, steps, operations and/or elements.

It will also be understood that when a layer (or film) is referred to as being ‘on’ another layer (or film) or substrate, it can be directly on the other layer (or film) or substrate, or third intervening layers (or films) may also be present.

It will be understood that, although the terms first, second, third etc. may be used herein to describe various regions, layers (or films), etc. these regions and layers should not be limited by these terms. These terms are only used to distinguish one region or layer (or film) from another region or layer (film). Thus, a first layer discussed below could be termed a second layer. Example embodiments embodied and described herein may include complementary example embodiments thereof. Like reference numerals refer to like elements throughout.

Unless otherwise defined, all terms used herein have the same meaning as commonly understood by one of ordinary skill in the art to which this inventive concept belongs.

In the present disclosure,

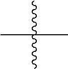

means a connected part.

In the present disclosure, a substituted or unsubstituted alkyl group may mean an alkyl group which is substituted or unsubstituted with at least one substituent selected from the group consisting of a hydrogen atom, a deuterium atom and an alkyl group. In addition, the exemplified substituents may be substituted or unsubstituted substituents. In the present disclosure, the substituent may be interpreted as at least one substituent selected from a monovalent substituent or a divalent substituent.

An analysis apparatus according to exemplary embodiments of the inventive concept will be explained.

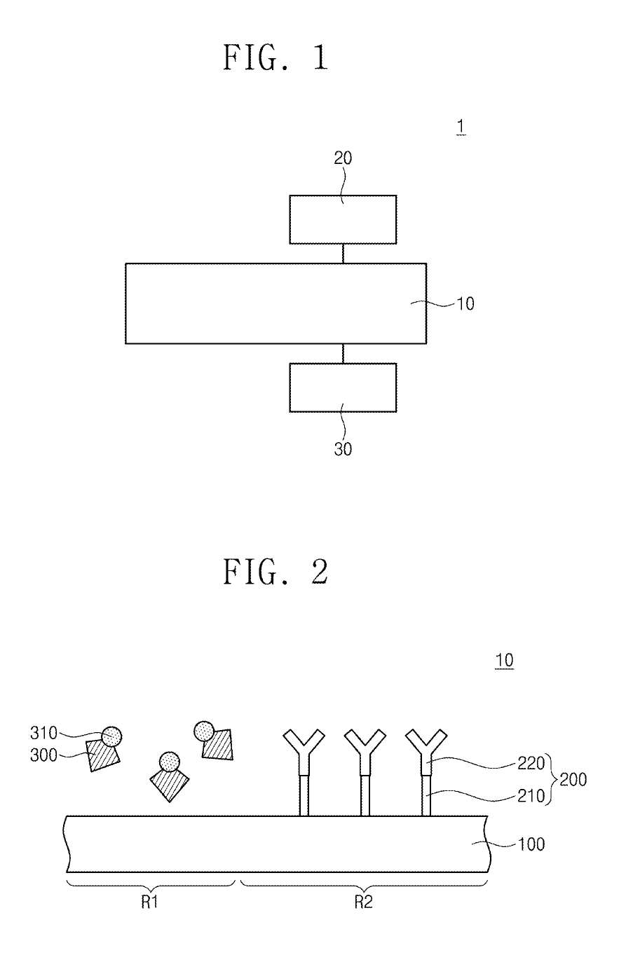

FIG. 1 is a diagram schematically illustrating an analysis apparatus according to exemplary embodiments.

Referring to FIG. 1, an analysis apparatus 1 may include a detection part 10, a light source part 20, and a sensing part 30. The analysis apparatus 1 may be used for analyzing a biomaterial. The biomaterial may include, for example, hormones such as cortisol. A biomaterial may be supplied to the detection part 10. The light source part 20 may supply light to the detection part 10. The detection part 10 may absorb light with a first wavelength. The sensing part 30 may measure the light with a first wavelength and transform the measured light into electrical signals. Though not shown, the analysis apparatus 1 may further include a control part and a display part. Hereinafter, the detection part 10 will be explained in more detail.

FIG. 2 is a cross-sectional view illustrating a detection part of an analysis apparatus according to exemplary embodiments. Hereinafter, overlapped contents with the above-explanation will be omitted.

Referring to FIG. 2, a detection part 10 may include a substrate 100, antibodies 300, and a capturing structure 200. The substrate 100 may be a well plate or a capillary. The substrate 100 is shown planar, but an embodiment of the inventive concept is not limited thereto. The substrate 100 may include at least one of plastic or glass. The substrate 100 may include a first region R1 and a second region R2.

The antibodies 300 may be supplied in the first region R1. The antibodies 300 may be physically adsorbed on the substrate 100. The antibodies 300 may not chemically bind to the substrate 100. The antibodies 300 may include, for example, antibodies against at least one among cortisol and the derivatives thereof. The antibodies 300 may have labels 310. The labels 310 may be combined with the antibodies 300. The labels 310 may include peroxidase enzymes. The peroxidase enzyme may include, for example, a horseradish peroxidase (HRP) enzyme. The antibodies 300 may be supplied in excessive quantity. The antibodies 300 may not be provided in the second region R2 of the substrate 100.

The capturing structure 200 may be provided in the second region R2 of the substrate 100. The capturing structure 200 may not be provided in the first region R1 of the substrate 100. The capturing structure 200 may include a linker 210 and a first antigen 220. The linker 210 may bind to the substrate 100. The bond between the substrate 100 and the linker 210 may be a covalent bond. The linker 210 may include an organic material. For example, the linker 210 may be represented by the following Formula 1:

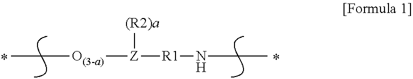

(in Formula 1, Z is one selected among silicon (Si) and carbon (C), R₁ includes at least one selected from —(CH₂)ₙ—, —(CH₂)ₘ—(CH₂CH₂)ₙ—, and —(CH₂)ₘ—(NH—CH₂CH₂)ₙ—, and R₂ is a substituted or unsubstituted alkyl group having 1 to 5 carbon atoms. “a” is an integer selected among 0, 1 and 2. “n” is an integer selected from 1 to 10. “m” is an integer selected from 0 to 10. # may mean a combined part with the substrate 100. * may mean a combined part with the first antigen 220.)

For example, the linker 210 may be represented by at least one of the following Formula 2a, Formula 2b or Formula 2c:

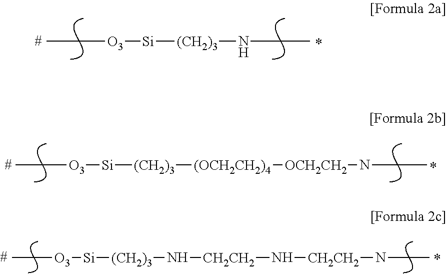

(in Formula 2a, Formula 2b and Formula 2c, # may mean a combined part with the substrate 100. * may mean a combined part with the first antigen 220.)

The first antigen 220 may bind to the linker 210. The antibodies 300 may be antibodies against the first antigen 220. The first antigen 220 may include at least one of cortisol or the derivatives thereof. For example, the first antigen 220 may include a material represented by the following Formula 3:

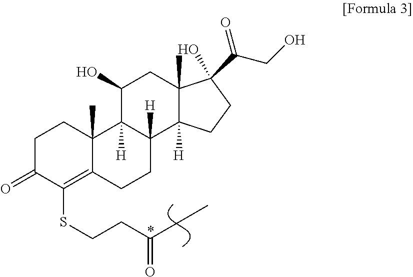

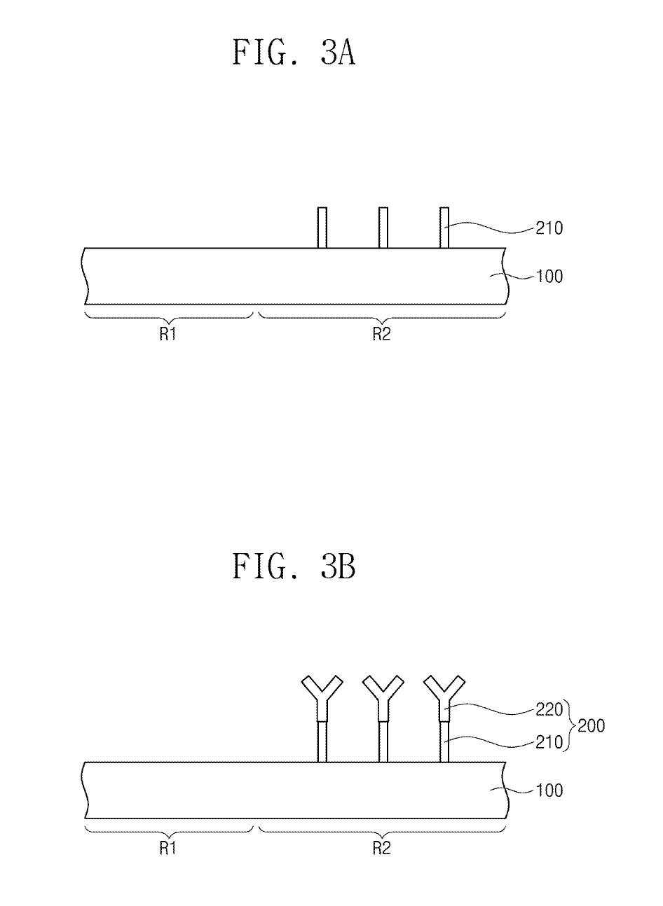

FIGS. 3A and 3B are cross-sectional views illustrating a method of manufacturing an analysis apparatus according to exemplary embodiments. Hereinafter, overlapped contents with the above-explanation will be omitted.

Referring to FIG. 3A, a linker 210 may be formed in the second region R2 of a substrate 100. According to exemplary embodiments, a substrate 100 may be prepared. A plasma treatment process may be conducted with respect to the substrate 100, and functional groups may be formed on the substrate 100. The plasma treatment process may include an oxygen plasma treatment process. The functional group may include a hydroxyl group (—OH).

A linker precursor (not shown) may be supplied onto the substrate 100. The linker precursor may be supplied into the second region R2 of the substrate 100, but may not be provided in the first region R1 of the substrate 100. The linker precursor may be represented by the following Formula 4a or Formula 4b:

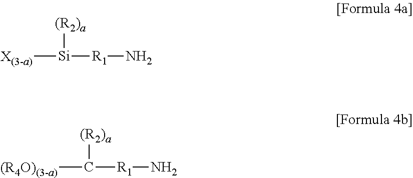

(in Formula 4a and Formula 4b, R₁ includes at least one selected from —(CH₂)ₙ—, —(CH₂)ₘ—(CH₂CH₂)ₙ—, or —(CH₂)ₘ—(NH—CH₂CH₂)ₙ—, and R₂ and R₄ are each independently a substituted or unsubstituted alkyl group having 1 to 5 carbon atoms. X may include any one selected from —F, —Cl, —Br, or —I. “a” may be an integer selected from any one of 0, 1 or 2. “n” may be an integer selected from any one of 1 to 10. “m” may be an integer selected from any one of 0 to 10.)

According to exemplary embodiments, the linker precursor may be represented by at least one of the following Formula 5a, Formula 5b or Formula 5c:

(CH₃O)₃—Si—(CH₂)₃—NH₂ [Formula 5a]

(CH₃O)₃—Si—(CH₂)₃—(OCH₂CH₂)₄OCH₂CH₂—NH₂ [Formula 5b]

(CH₃O)₃—Si—(CH₂)₃—NH—CH₂CH₂—NH—CH₂CH₂—NH₂ [Formula 5c]

If the linker precursor is represented by Formula 5b, the linker precursor may be prepared by the following Reaction 2:

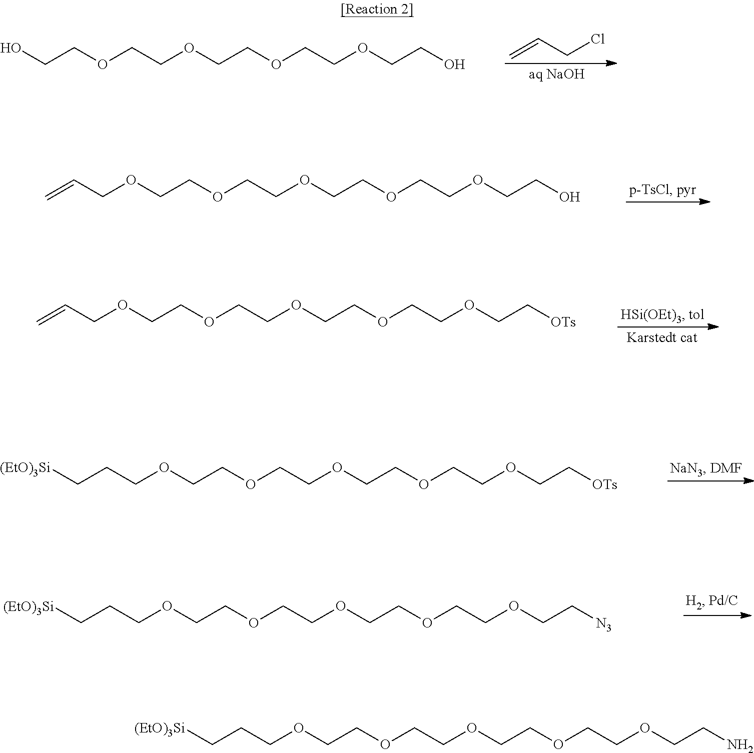

(in Reaction 2, Et is CH₃CH₂—, DMF is dimethylformamide, p-TsCl is p-toluenesulfonyl chloride, Ts is p-toluenesulfonyl, and pyr is pyridine. tol is toluene. Karstedt cat may include Pt as a catalyst of hydrosilylation reaction. In an embodiment, Karstedt cat may include a material represented by C₂₄H₅₄O₃Pt₂Si₆.)

The linker precursor may react with the functional group of the substrate 100 to form the linker 210. The linker 210 may bind to the substrate 100. If the linker precursor is represented by Formula 4a, X of Formula 4a may react with the functional group of the substrate 100. If the linker precursor is represented by Formula 4b, (R₄O) of Formula 4b may react with the functional group of the substrate 100. The reaction of the functional group of the substrate 100 and the linker precursor may be conducted according to the following Reaction 3:

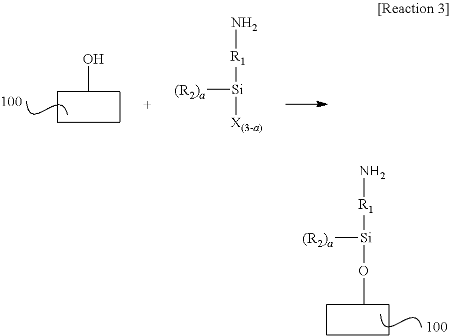

(in Reaction 3, R₁ includes at least one selected from —(CH₂)ₙ—, —(CH₂)ₘ—(CH₂CH₂)ₙ—, or —(CH₂)ₘ—(NH—CH₂CH₂)ₙ—, and R₂ is a substituted or unsubstituted alkyl group having 1 to 5 carbon atoms. “a” may be an integer selected from any one of 0, 1 or 2. “n” may be an integer selected from any one of 1 to 10. “m” may be an integer selected from any one of 0 to 10.)

Referring to FIG. 3B, a capturing structure 200 may be formed in the second region R2 of the substrate 100. According to exemplary embodiments, the first antigen 220 may be supplied onto the substrate 100. The first antigen 220 may include cortisol and cortisol derivatives. The first antigen 220 may be synthesized by the following Reaction 4:

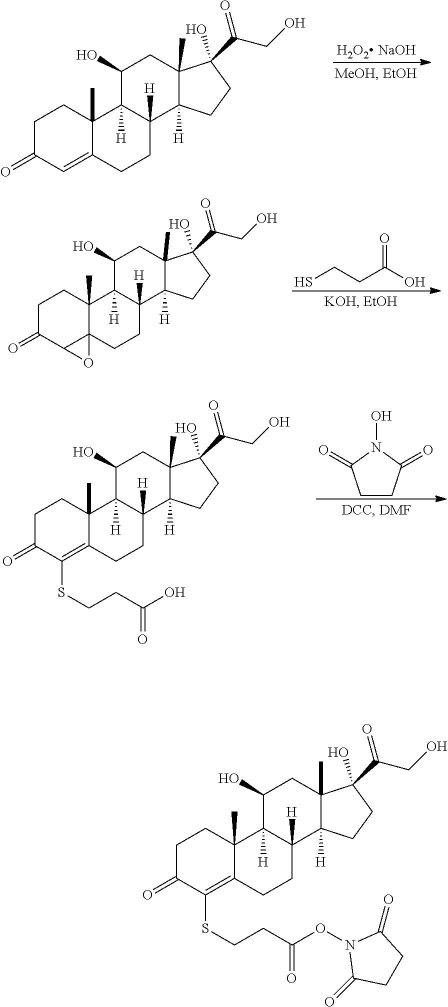

(in Reaction 4, MeOH is CH₃OH, EtOH is CH₃CH₂OH, DCC is dicyclohexylcarbodiimide, and DMF is dimethylformamide.)

A reaction may be conducted between the first antigen 220 and the linker 210. For example, the reaction between the first antigen 220 and the linker 210 may be conducted by the following Reaction 5:

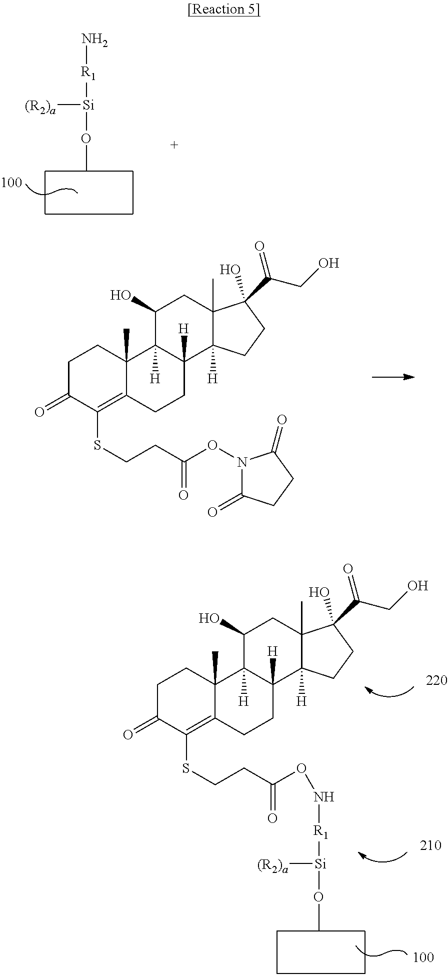

(in Reaction 5, R₁ includes at least one selected from —(CH₂)ₙ—, —(CH₂)ₘ—(CH₂CH₂)ₙ—, or —(CH₂)ₘ—(NH—CH₂CH₂)ₙ—, and R₂ is a substituted or unsubstituted alkyl group having 1 to 5 carbon atoms. “a” is an integer selected from any one of 0, 1 or 2. “n” is an integer selected from any one of 1 to 10. “m” is an integer selected from any one of 0 to 10.)

Through the reaction, the first antigen 220 may bind to the linker 210. The bond between the first antigen 220 and the linker 210 may include a covalent bond. Accordingly, the capturing structure 200 may be formed.

Referring to FIG. 2 again, the antibodies 300 may be supplied into the first region R1 of the substrate 100. The antibodies 300 may be formed by a freezing process. The antibodies 300 may be physically adsorbed onto the substrate 100. The antibodies 300 may have labels 310. As described so far, the analysis apparatus 1 may be manufactured. Alternatively, after supplying the antibodies 300 into the first region R1 of the substrate 100, the capturing structure 200 may be formed.

Hereinafter, a method of analyzing a biomaterial according to exemplary embodiments will be explained.

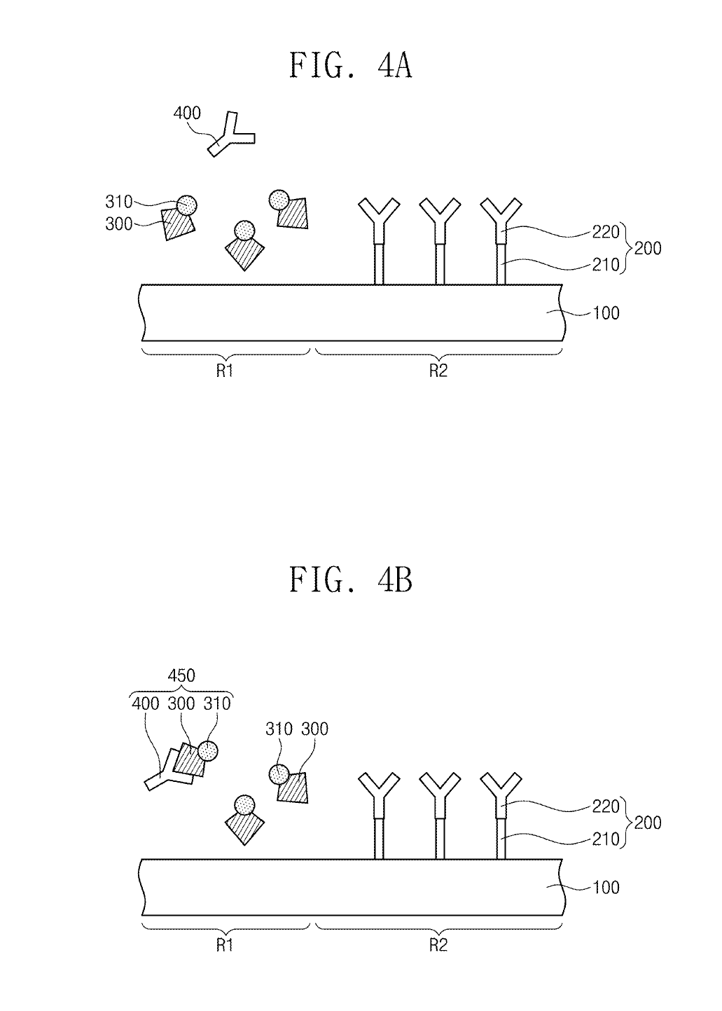

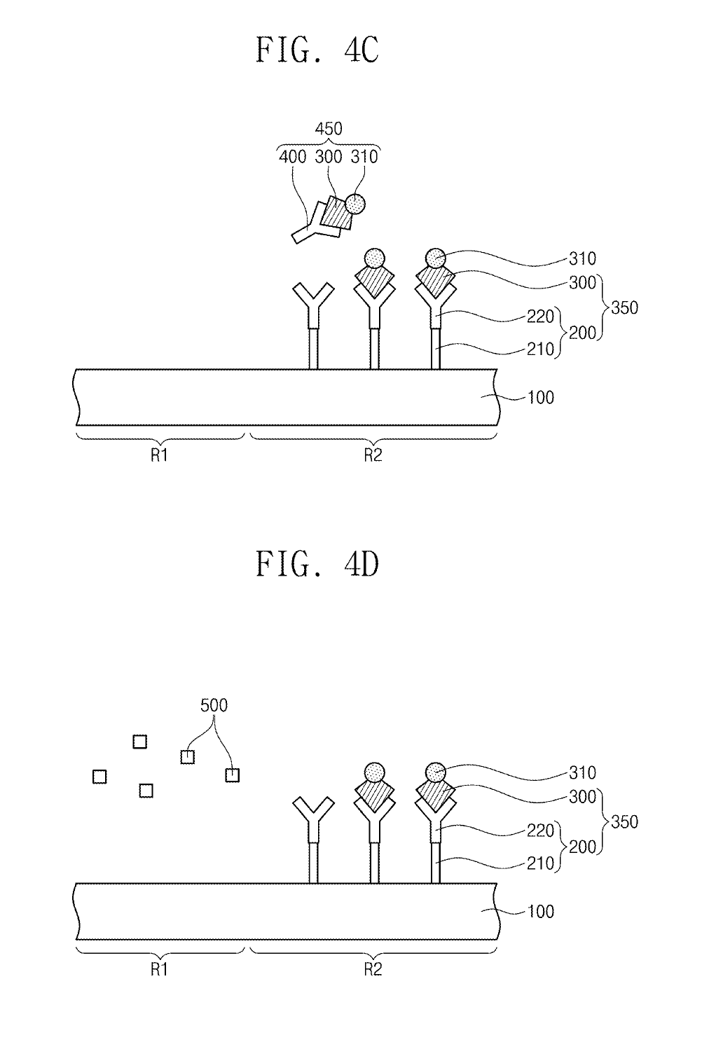

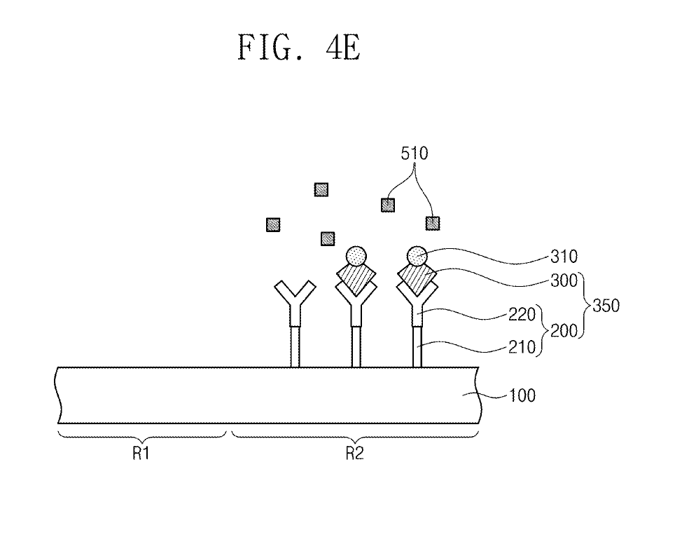

FIGS. 4A to 4E are cross-sectional views illustrating a method of analyzing a biomaterial according to exemplary embodiments. Hereinafter, overlapped contents with the above-explanation will be omitted.

Referring to FIG. 4A, a sample may be supplied to a detection part 10 of an analysis apparatus 1. The detection part 10 may be substantially the same as the detection part 10 which has been explained referring to FIGS. 1 and 2. The sample may be supplied into the first region R1 of the substrate 100. Blood, saliva, or urine may be used as the sample. The sample may include a second antigen 400. The second antigen 400 may be a material to be analyzed. The second antigen 400 may have a small molecular weight (for example, about 362 g/mol). The second antigen 400 may be supplied in the sample in small quantity. The second antigen 400 may function as an antigen against the antibodies 300. The second antigen 400 may include, for example, at least one of cortisol or cortisol derivatives. The sample may further include a solvent.

Referring to FIG. 4B, a first reaction may be conducted between a portion of the antibodies 300 and the second antigen 400. The first reaction is antigen-antibody reaction and may be specific reaction. Through the first reaction, an antigen-antibody complex 450 may be formed. The antigen-antibody complex 450 may include the second antigen 400 and any corresponding one among the antibodies 300. The number of the second antigens 400 may be smaller than the number of the antibodies 300. After the first reaction, another portion of the antibodies 300 may not react but may remain. Hereinafter, in FIGS. 4C to 4E, the antibodies 300 may indicate another portion of the antibodies 300, that is, the remaining antibodies 300.

Referring to FIG. 4C, the antibodies 300 may move to the second region R2 of the substrate 100. The antibodies 300 may move by a solvent. A second reaction may be conducted between the antibodies 300 and the first antigen 220. The second reaction is antigen-antibody reaction and may be specific reaction. The second reaction may be in competitive relation with the first reaction of FIG. 4B. Through the second reaction, the antibodies 300 may bind to the first antigen 220 of the capturing structure 200. The antigen-antibody complex 450 may not be captured by the capturing structure 200. After that, a washing process is conducted with respect to the substrate 100 to remove the antigen-antibody complex 450.

Referring to FIG. 4D, a colorimetric substrate solution may be supplied onto the substrate 100. The colorimetric substrate solution may include a colorimetric material 500. The colorimetric substrate solution may be clear. 3,3′,5,5′-tetramethylbenzidine may be used as the colorimetric material 500. In this case, the colorimetric substrate solution may include 3,3′,5,5′-tetramethylbenzidine (hereinafter, TBM) and hydrogen peroxide (H₂O₂). The reaction of the colorimetric-material 500 may be hard to be conducted at room temperature without a catalyst due to high activation energy.

Referring to FIG. 4E, the colorimetric substrate solution may move from the first region R1 to the second region R2 of the substrate 100. The colorimetric substrate solution may make contact with the binding structure 350. The labels 310 may act as a catalyst of the colorimetric-material 500. The colorimetric-material 500 may react by the labels 310 to form colored products 510. The colored products 510 absorb light with a first wavelength to show color. If the TBM is used as the colorimetric-material 500, the TBM may react with hydrogen peroxide under a peroxidase enzyme to form the colored products 510.

The colored products 510 may absorb, for example, light of about 650 nm or about 950 nm. In another embodiment, a quenching reagent may be further supplied onto the substrate 100. The quenching reagent may include, for example, an acid such as sulfuric acid. In this case, the colored products 510 may absorb light having about 450 nm. In another embodiment, the colorimetric-material 500 may include 3,3′-diaminobenzidine or 2,2′-azino-bis(3-ethlbenzothiazoline-6-sulphonic acid.

Referring to FIG. 4E together with FIG. 1, light may be supplied into the second region R2 of the substrate 100. The light with the first wavelength may be absorbed by the colored products 510. The first wavelength may be about 950 nm, about 650 nm, or about 450 nm. The sensing part 30 may measure the light of the first wavelength, and the light of the first wavelength absorbed by the colored products 510 may be calculated and analyzed. Accordingly, the amount of the antibodies 300 may be measured. The second antigen (400 in FIG. 4A) may have a small molecular weight (for example, about 362 g/mol). Due to the small molecular weight of the second antigen 400, the quantitative analysis of the second antigen 400 may become difficult. Since the second antigen 400 is supplied in small quantity, the quantitative analysis of the second antigen 400 may become even further difficult. The antibodies 300 may be supplied into the first region R1 of the substrate 100 in excessive quantity. The quantitative analysis of the antibodies 300 may be easier than the quantitative analysis of the second antigen 400. From the amount measured of the antibodies 300, the supplied second antigen 400 may be calculated. According to exemplary embodiments, the quantitative analysis of the antibodies 300 may be easily conducted using the first reaction and the second reaction.

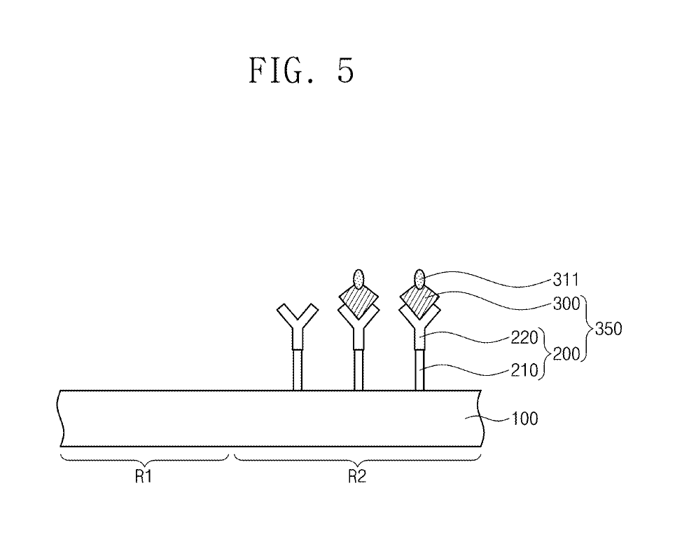

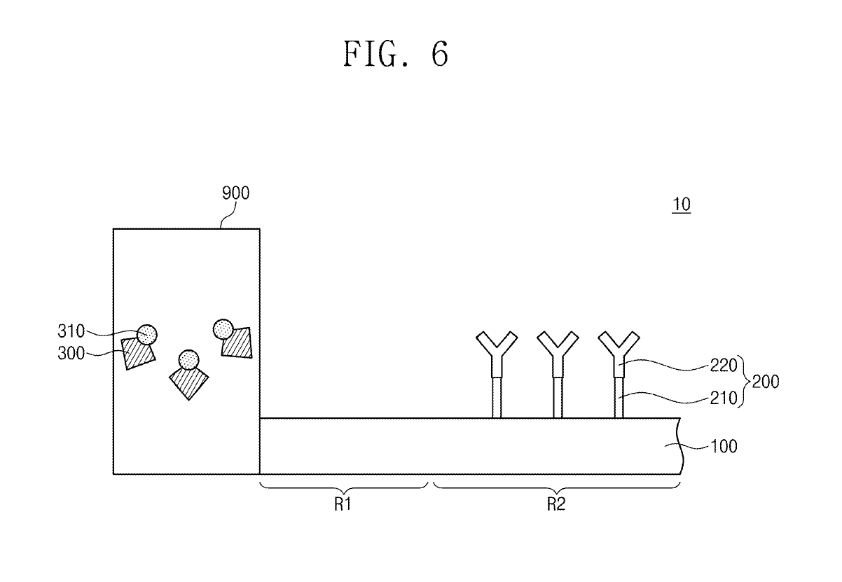

FIG. 5 is a cross-sectional view for explaining an analyzing method according to another embodiment. Hereinafter, overlapped contents with the above-explanation will be omitted for the simplification of explanation.

Referring to FIG. 5, labels 311 may include a fluorescence material, a chemifluorescence material such as luminol, or gold nanoparticles. The labels 311 may bind to the antibodies 300. The antibodies 300 may be combined with the capturing structure 200 to form a binding structure 350. The supplying method of a sample and the formation of the binding structure 350 may be substantially the same as those explained referring to FIGS. 4A to 4C. The supplying step of the colorimetric substrate solution, explained referring to FIG. 4D, may be omitted. Light may be absorbed by the labels 311. The sensing part 30 may measure the light absorbed and quantitatively analyze the antibodies 300. From the amount of the antibodies 300, the second antigen 400 may be quantitatively analyzed.

Hereinafter, an analysis apparatus and a method of analyzing a biomaterial using the same according to exemplary embodiments will be explained.

FIG. 6 is a cross-sectional view illustrating a detection part of an analysis apparatus according to exemplary embodiments. Hereinafter, overlapped contents with the above-explanation will be omitted.

Referring to FIGS. 1 and 6, a detection part 10 may include a filter 900 in addition to a substrate 100. The substrate 100 may include a first region R1 and a second region R2. A capturing structure 200 may be provided in the second region R2 of the substrate 100. The capturing structure 200 may include a linker 210 and a first antigen 220.

The filter 900 may be provided on one side of the substrate 100. The filter 900 may be adjacent to the first region R1 of the substrate 100. The antibodies 300 may be supplied into the filter 900. The antibodies 300 may be adsorbed in the filter 900. The antibodies 300 may have labels 310. The linker 210, the first antigen 220, the antibodies 300 and the labels 310 may include the same materials as those explained in the embodiment of FIG. 2. In another embodiment, the labels 310 may include the same materials as those explained in FIG. 5.

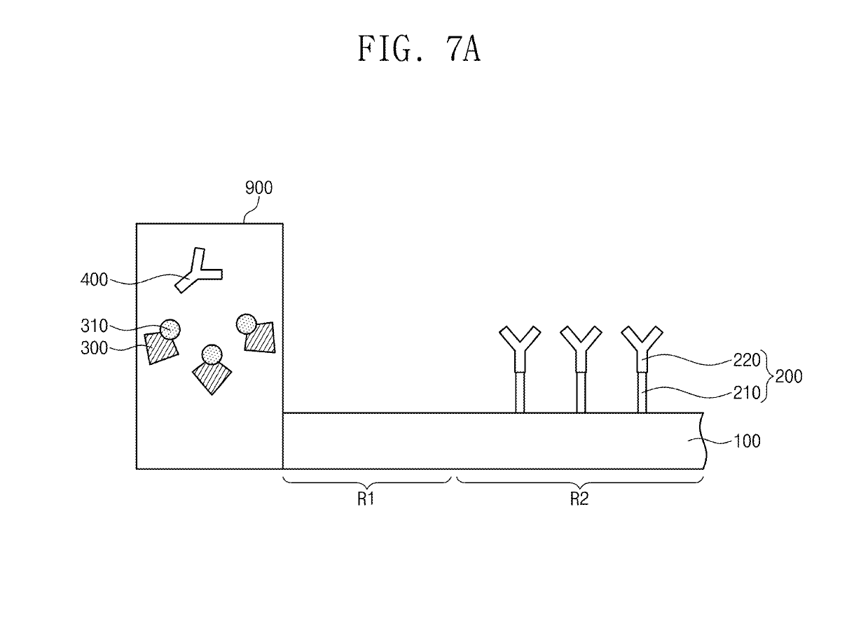

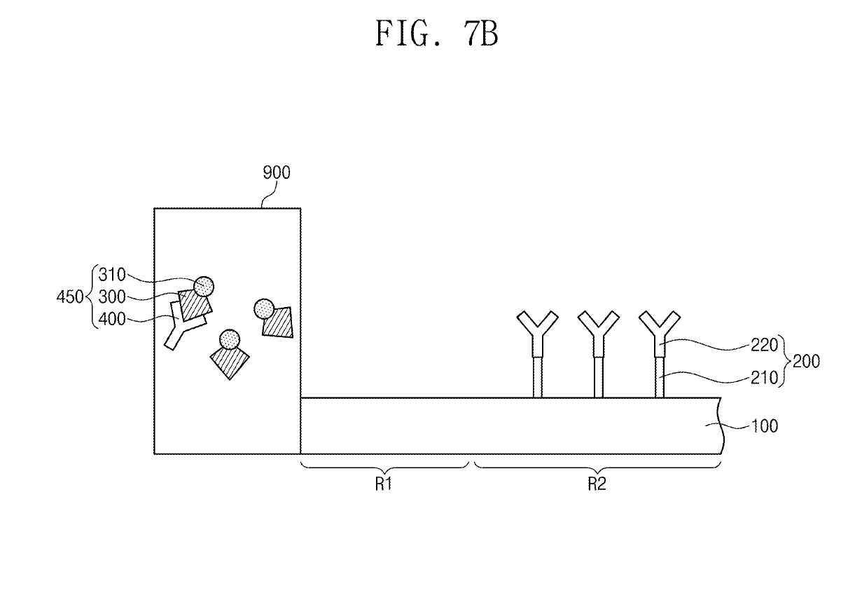

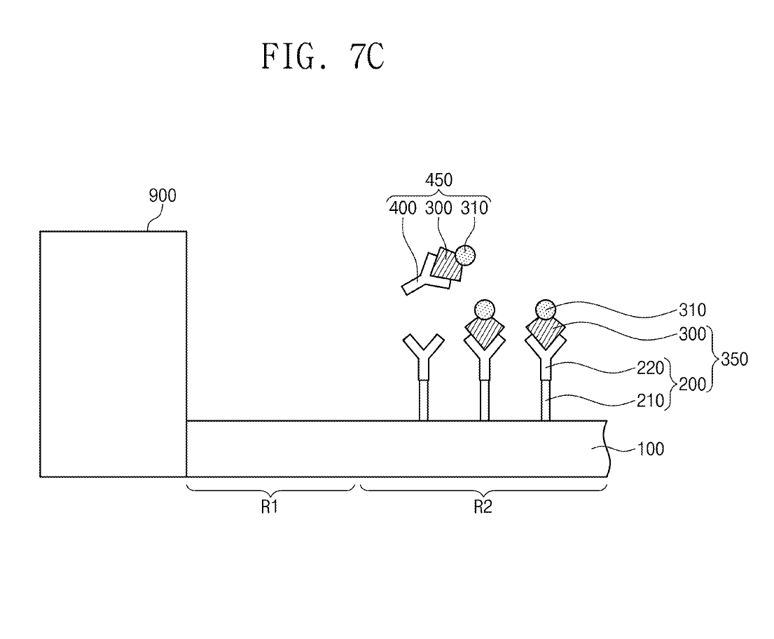

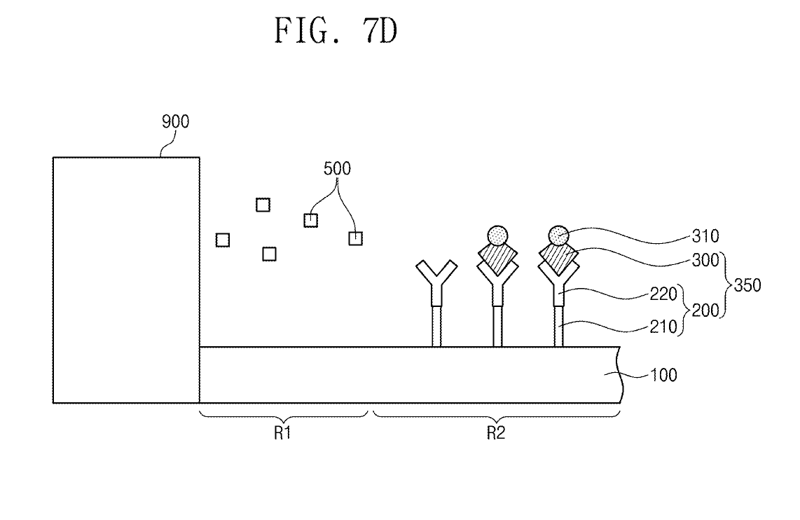

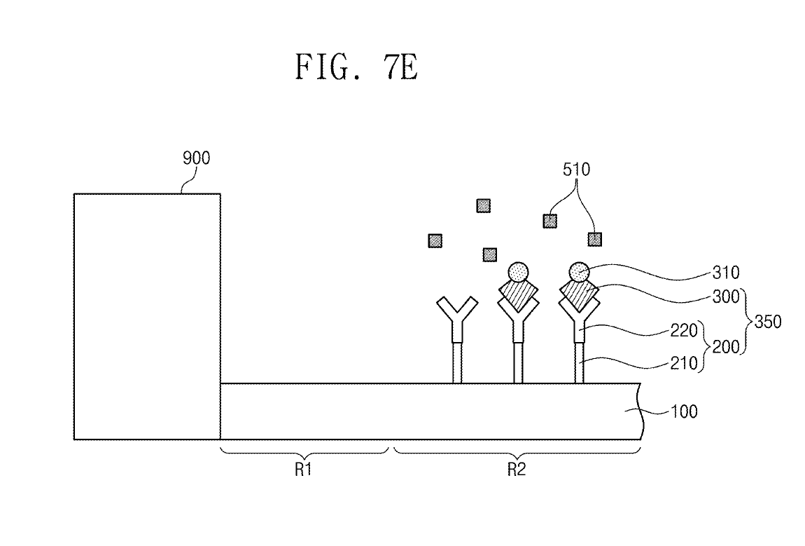

FIGS. 7A to 7E are cross-sectional views illustrating a method of analyzing a biomaterial according to exemplary embodiments. Hereinafter, overlapped contents with the above-explanation will be omitted.

Referring to FIG. 7A, a sample may be supplied into a filter 900 of a detection part 10. The detection part 10 may be substantially the same as that explained in FIG. 6. The sample may include a second antigen 400 and a solvent. The second antigen 400 may be the same as the second antigen 400 as that explained in FIG. 4A.

Referring to FIG. 7B, a first reaction of a portion of the antibodies 300 and the second antigen 400 may be conducted to form an antigen-antibody complex 450. After the first reaction, another portion of the antibodies 300 may not participate in the first reaction. Hereinafter, in FIGS. 7C to 7E, the antibodies 300 may mean another portion of the antibodies 300, that is, the remaining antibodies 300.

Referring to FIG. 7C, an antigen-antibody complex 450 and the antibodies 300 may move into the second region R2 of the substrate 100. A second reaction of the antibodies 300 and a first antigen 220 may be conducted. Through the second reaction, the antibodies 300 may be combined with the first antigen 220 of the capturing structure 200. The antigen-antibody complex 450 may not be captured by the capturing structure 200. After that, the antigen-antibody complex 450 may be removed by a washing process.

Referring to FIG. 7D, a colorimetric-material 500 may be supplied via the filter 900 onto the substrate 100. The colorimetric-material 500 may include the same materials as those explained in FIG. 5D.

Referring to FIG. 7E and FIG. 1, the colorimetric-material 500 may move from the first region R1 to the second region R2 of the substrate 100. The colorimetric-material 500 may make contact with the binding structure 350. The colorimetric-material 500 may react by labels 310 to form colored product 510. Light may be supplied into the second region R2 of the substrate 100. Light of a first wavelength may be absorbed by the colored products 510. A sensing part 30 may measure the light of a first wavelength, and the antibodies 300 may be quantitatively analyzed. From the amount of the antibodies 300, the second antigen 400 may be quantitatively analyzed.

According to another embodiment, the labels 310 may include a fluorescence material, a chemifluorescence material, or gold nanoparticles. In this case, the supplying step of the colorimetric substrate solution explained in FIG. 7D may be omitted. Light may be absorbed or reflected by the labels 310. The sensing part 30 may measure the light of a first wavelength and may quantitatively analyze the antibodies 300. From the amount of the binding structure 350, the second antigen 400 may be quantitatively analyzed.

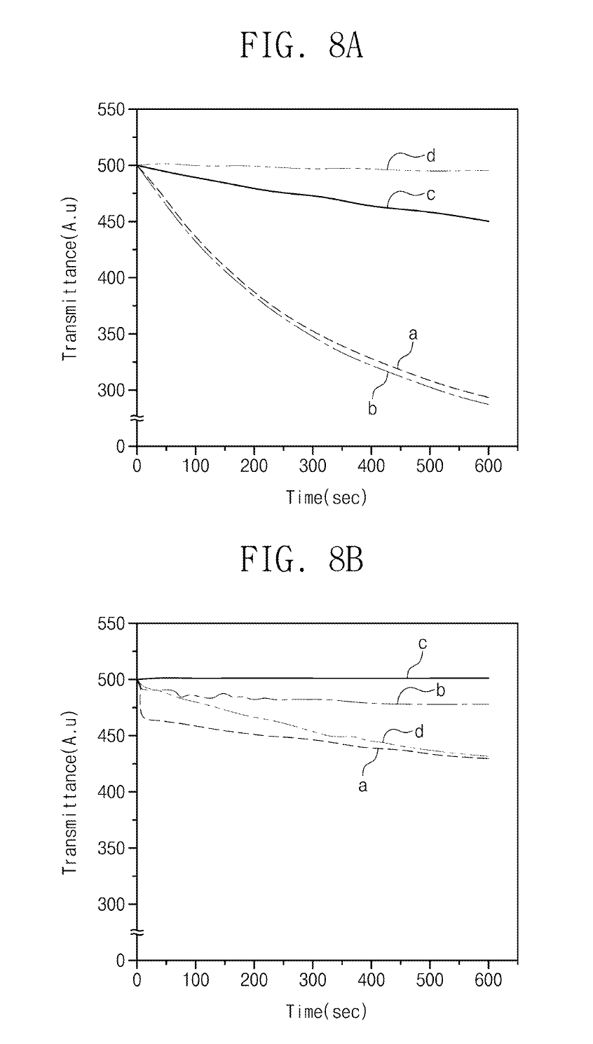

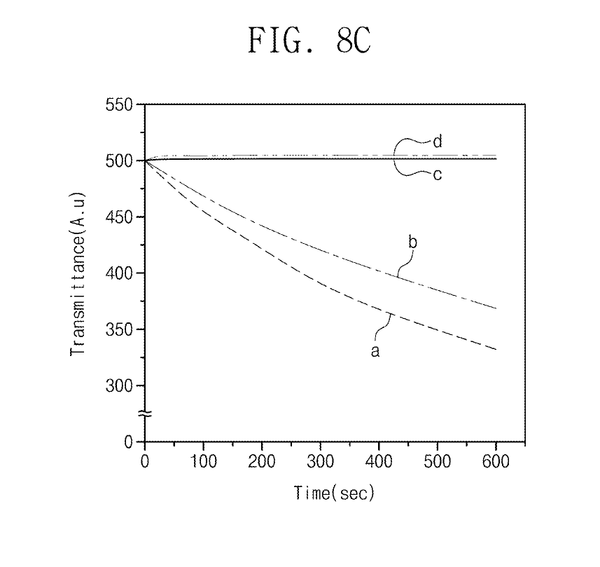

FIGS. 8A to 8C illustrate the measured results of transmittance using linkers represented by Formula 5a to Formula 5c, respectively. In FIGS. 8A to 8C, transmittance at about 650 nm was measured. Graphs a, b, c and d are analysis results with the concentration of the second antigen 400 of about 0 ng/mol, about 1 ng/mol, about 10 ng/mol and about 50 ng/mol, respectively. Cortisol was used as a second antigen. The unit of the y-axis is an optional value. Hereinafter, embodiments of the inventive concept will be explained referring to FIGS. 2 and 3A to 3E, together.

Referring to FIGS. 8A to 8C, it may be found that the transmittance decreases with the increase of the concentration of the second antigen 400. From the transmittance, the second antigen 400 may be quantitatively analyzed.

Referring to FIG. 8A, it may be found that if a linker 210 represented by Formula 5a was used, the quantitative analysis of cortisol with high concentrations (b, c and d) showed high sensitivity. Referring to FIG. 8B, it may be found that if a linker 210 represented by Formula 5b was used, the quantitative analysis of cortisol with middle concentrations (b and c) showed high sensitivity. Referring to FIG. 8C, it may be found that if a linker 210 represented by Formula 5c was used, the quantitative analysis of cortisol with low concentrations (a, b and c) showed high sensitivity. The linker 210 may be selected according to the properties of a material to be analyzed (for example, concentration or kind).

According to the inventive concept, a second antigen may have a small molecular weight. The second antigen may be supplied in a sample in small quantity. Through a first reaction and a second reaction, antibodies captured in a binding structure may be quantitatively analyzed. From the amount of the antibodies, the second antigen may be quantitatively analyzed easily. The quantitative analysis of the second antigen may show high sensitivity and accuracy.

Although the exemplary embodiments of the present invention have been described, it is understood that the present invention should not be limited to these exemplary embodiments but various changes and modifications can be made by one ordinary skilled in the art within the spirit and scope of the present invention as hereinafter claimed.

What is claimed is:

1. A method of analyzing a biomaterial, the method comprising:
preparing a substrate including a first region and a second region, antibodies being disposed in the first region of the substrate, a capturing structure being provided in the second region of the substrate, the capturing structure comprising a linker which binds to the substrate and a first antigen which binds to the linker;
supplying a second antigen onto the substrate to conduct a first reaction of the second antigen and a portion of the antibodies; and
conducting a second reaction of another portion of the first antigen and the antibodies after conducting the first reaction, to form a binding structure,
wherein the binding structure comprises at least one of the linker, the first antigen or the antibodies.

2. The method of analyzing a biomaterial of claim 1, further comprising:
supplying a colorimetric-material solution onto the substrate to form a colored product;
supplying light onto the substrate; and
analyzing light absorbed by the colored product.

3. The method of analyzing a biomaterial of claim 2, wherein the antibodies have labels, and the labels comprise a peroxidase enzyme.

4. The method of analyzing a biomaterial of claim 3, wherein the colorimetric-material solution comprises 3,3′,5,5′-tetramethylbenzidine and hydrogen peroxide (H₂O₂).

5. The method of analyzing a biomaterial of claim 1, wherein the first antigen comprises at least one of cortisol or cortisol derivatives, and the second antigen comprises at least one of cortisol or cortisol derivatives.

6. The method of analyzing a biomaterial of claim 1, wherein the linker is represented by the following Formula 1: 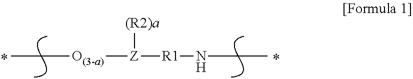
(in Formula 1, Z is one selected among silicon (Si) and carbon (C), R₁ comprises at least one selected from —(CH₂)ₙ—, —(CH₂)ₘ—(CH₂CH₂)ₙ—, and —(CH₂)ₘ—(NH—CH₂CH₂)ₙ—, R₂ is a substituted or unsubstituted alkyl group having 1 to 5 carbon atoms, “a” is an integer selected among 0, 1 and 2, “n” is an integer selected from 1 to 10, and “m” is an integer selected from 0 to 10).

7. A method of analyzing a biomaterial, the method comprising:
preparing an analysis apparatus comprising a filter and a substrate, antibodies being provided in the filter, a capturing structure being provided on the substrate, the capturing structure comprising a linker which binds to the substrate and a first antigen which binds to the linker;
supplying second antigens into the filter to conduct a first reaction of the second antigen and a portion of the antibodies;
moving another portion of the antibodies onto the substrate after conducting the first reaction; and
forming a binding structure via a second reaction of the first antigen another and portion of the antibodies,
wherein the binding structure comprises at least one of the linker, the first antigen, or the antibodies.

8. The method of analyzing a biomaterial of claim 7, further comprising:
supplying a colorimetric-material solution onto the substrate to form a colored product;
supplying light onto the substrate; and
analyzing light absorbed by the colored product to quantitatively analyze the second antigen.

9. The method of analyzing a biomaterial of claim 7, wherein the first antigen comprises at least one of cortisol or cortisol derivatives, and the second antigen comprises at least one of cortisol or cortisol derivatives.

10. The method of analyzing a biomaterial of claim 7, wherein the linker is represented by the following Formula 1: 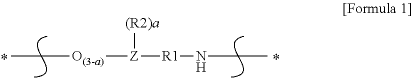
(in Formula 1, Z is one selected among silicon (Si) and carbon (C), R₁ comprises at least one selected from —(CH₂)ₙ—, —(CH₂)ₘ—(CH₂CH₂)ₙ—, and —(CH₂)ₘ—(NH—CH₂CH₂)ₙ—, R₂ is a substituted or unsubstituted alkyl group having 1 to 5 carbon atoms, “a” is an integer selected among 0, 1 and 2, “n” is an integer selected from 1 to 10, and “m” is an integer selected from 0 to 10).

11. The method of analyzing a biomaterial of claim 7, wherein the substrate comprises a well plate or a capillary.
